(19) United States
(12) Patent Application Publication
(10) Pub. No.: US 2007/0118174 A1
(43) Pub. Date: May 24, 2007

(54) LAPAROSCOPIC SURGICAL CLAMP AND SUTURING METHODS

(76) Inventor: David Z. J. Chu, San Gabriel, CA (US)

Correspondence Address: CHAN LAW GROUP LC, 1055 W. 7TH ST,, SUITE 1880, LOS ANGELES, CA, 90017, US

(21) Appl. No.: 11/560,730

(22) Filed: Nov. 16, 2006

(51) Int. Cl. A61B 17/00; A61B 17/08

(52) U.S. Cl. 606/207; 606/157

(57) ABSTRACT

A method of occluding a patient's organ tissue during a minimally invasive surgical procedure is disclosed. The method includes providing a laparoscopic surgical clamp; passing distal end of surgical clamp into an opening of patient's body cavity; probing patient's tissue by moving first set and second set of double jaws of the surgical clamp between opened and closed positions; selecting tissue appropriate for being occluded with surgical clamp and suturing; clamping tissue with surgical clamp; passing at least one elongated suture into patient's body cavity with laparoscopic grasper; threading suture through a plurality of fenestration on at least one of upper portions of double jaws; puncturing patient's tissue with suture; threading suture through fenestration of at least one of the lower portions of double jaws; and clipping each end of suture on upper and lower portions of double jaws for securing the patient's tissue in a compressed position.

### BACKGROUND OF THE INVENTION

1. Field of the Invention

The present invention relates to the field of medical devices, and more specifically to methods for occluding and suturing a patient's organ tissue using a laparoscopic surgical clamp in conjunction with sutures during minimally invasive surgical procedures. The laparoscopic surgical clamp is particularly beneficial in that it allows for resections of liver lesions of different sizes and anatomical locations and lesions in close proximity to the major liver vessels. A novel suturing method to complement this laparoscopic technique as well as other ligation techniques is disclosed and is ideal for liver and other solid organs in order to achieve hemostasis.

2. Description of the Prior Art

Surgical clamps that use various types of clamping jaws and clamping configurations (i.e., curved, straight, and the like) are well known in the medical device art. In particular, large tissue clamps to accommodate substantial vessel or tissue compression for open surgical procedures such as laparotomies are used for major vascular procedures on the aorta or venal cava with, for example, Satinsky or Sehrt's clamps, and for gynecological procedures with Haney clamps, which also have a serrated mechanism at the clamp tip to prevent tissue slippage. Gastric clamps such as the Payrs clamp combines clamping and crushing of tissues for ease of transection. Large liver clamps of the Lin type have been used for hemostatic purposes. The Chu liver clamp was designed as a Lin liver clamp except it has larger and curved jaws.

For laparoscopic approaches, examples of vascular clamps that have been developed are disclosed in Anderson, et al., Patent Publication No. 2005/0251184, Douglas, et al., Publication No. 2005/0165429, Gold, et al., Publication No. 2003/0212435, and Schwarz, et al., Patent Publication No. 2005/0147585. Similarly, examples of sutures and suture methods that have been developed for use in minimally invasive surgical procedures are disclosed in Nguyen, et al., Patent Publication No. 2003/0191481, and Nobles, et al., U.S. Pat. No. 6,911,034.

In the minimally invasive setting, peripheral liver masses are currently tackled by the hand assistance method and other liver parenchyma transection techniques. The more centrally located liver nodules and masses present a unique challenge and most are resected using open surgical techniques. With the introduction of minimally invasive surgical procedures in recent years, surgical sites have decreased in size, and obstruction by large, surgical clamps of the surgeon's field of visibility or access to other locations at the surgical site have become problematic. Furthermore, while open liver resections have several techniques to control parenchymal bleeding from the liver, laparoscopic liver resections are primarily limited by the inability to control bleeding.

In addition, liver ligatures are difficult to apply by laparoscopic means. Liver sutures by their very nature are required to be large in terms of tissue bite for both vascular and bile duct control and hepatic parenchymal compression. In the open surgical procedure, the needles and sutures are ideally 1-2 inches in diameter and the caliber of sutures are either 0 or #1. Sutures of this size are difficult to introduce and maneuver within the minimally invasive body cavity.

It would be desirable to have methods for occluding and suturing a patient's organ tissue using a low-profile surgical clamp design and sutures that can be introduced into the abdominal cavity through a small port for use in minimally invasive surgical procedures. It would be also desirable to have methods for occluding and suturing a patient's organ tissue using a low-profile surgical clamp design and sutures that can be applied to internal organs, such as the liver, lung, kidney, and spleen, during minimally invasive surgical procedures. It would be further desirable to have a method for occluding or suturing a patient's organ tissue using a combination of a laparoscopic surgical clamp and sutures that will allow resections of liver lesions of different sizes and anatomical locations and lesions in close proximity to the major liver vessels. The aforementioned methods complement the technique of laparoscopic liver resection and other ligation techniques to achieve hemostasis. The methods overcome the aforementioned shortcomings by ease of passage of sutures through ports and ease of application in minimally invasive surgical procedures as the suture serves as an introducing needle as well.

### SUMMARY OF THE INVENTION

The present invention provides a method of occluding a patient's organ tissue during a minimally invasive surgical procedure. In a first aspect, the method includes providing a laparoscopic surgical clamp. The laparoscopic surgical clamp may be applied to various internal organs of a patient, including the liver, lung, spleen, and kidney. The laparoscopic surgical clamp used in combination with the method of the present invention includes an elongated shaft having a proximal end and a distal end. A first set and a second set of double jaws are connected to the distal end of the elongated shaft and moveable relative to one another. The first set and second set of double jaws include an opened position in which the first set and second set of double jaws are separated and substantially parallel to one another and a closed position in which the first set and second set of double jaws are adjacent and substantially parallel to one another.

At least one articulation segment is located on each jaw of the first set and second set of double jaws such that the jaws may be positioned in either a U- or V-shaped configuration when in an articulated position. A plurality of fenestration is located on at least one jaw of the first set and second set of double jaws allowing suturing through the first set and second set of double jaws.

The laparoscopic surgical clamp further includes a double hand assembly operatively connected to at least one of the first set and second set of double jaws such that the jaws may move between the opened and closed positions relative to the motion of the double hand assembly. The double handle assembly has a locking mechanism allowing ease of engagement of the first set and second set of double jaws between the opened and closed positions.

A distal end of the laparoscopic surgical clamp is passed into an opening of a patient's body cavity. The patient's organ tissue is probed by moving the first set and second set of double jaws between the opened and closed positions. Appropriate tissue is selected for being occluded with the laparoscopic surgical clamp and suturing. The patient's tissue is clamped with the laparoscopic surgical clamp.

At least one elongated suture is passed into the patient's body cavity with a laparoscopic grasper. The elongated suture is threaded through the plurality of fenestration of at least one of an upper portion of the first set and second set of double jaws. The patient's organ tissue is punctured with the elongated suture. The elongated suture is threaded through the plurality of fenestration of at least one of a lower portion of the first set and second set of double jaws. Each end of the at least one elongated suture is clipped on the upper and lower portions of the first set and second set of double jaws for securing the patient's tissue in a compressed position.

The suturing remains in place for hemostasis after resection of the patient's organ tissue and removal of the laparoscopic surgical clamp. The suture used in accordance with the present invention is configured as a tube-like member having an opening therein allowing compression and fixation by a plurality of clips applied across the suture. The suture includes a pointed end for puncturing through the patient's solid organ parenchyma. The suture is capable of puncturing through the entire thickness of the patient's tissue allowing closure and pressure on the same tissue plane. The suture may be fabricated from an absorbable material such as a polyglycolic polymer and is about 1.5-2 mm in diameter and about 10 cm in length.

Further compression of the suture against the patient's organ tissue may be achieved by reapplying the clip and cutting off excess length of the suture and discarding the earlier clip. The suture material may be cut by laparoscopic scissors and be adjusted to a desired length. The suture end is expandable allowing fixation within the parenchyma of the patient's solid organ. The suture may assume a curved configuration allowing closure and pressure on same tissue plane. Each end of the elongated suture on the upper and lower portions of the first set and second set of double jaws is clipped with a clip applicator.

In another aspect of the present invention, a method is provided for occluding a patient's organ tissue during a minimally invasive surgical procedure. The method includes providing a laparoscopic surgical clamp. A distal end of the laparoscopic surgical clamp is passed into an opening of a patient's body cavity. The patient's organ tissue is probed by moving a first set and second set of double jaws of the laparoscopic surgical clamp between an open and closed position. Appropriate tissue is selected for being occluded with the laparoscopic surgical clamp and suturing. The patient's tissue is clamped with the laparoscopic surgical clamp.

At least one elongated suture is passed into the patient's body cavity with a laparoscopic grasper. The elongated suture is threaded through the plurality of fenestration of at least one of an upper portion of the first set and second set of double jaws. The patient's organ tissue is punctured with the elongated suture. The elongated suture is threaded through the plurality of fenestration of at least one of a lower portion of the first set and second set of double jaws. Each end of the at least one elongated suture is clipped on the upper and lower portions of the first set and second set of double jaws with a clip applicator for securing the patient's tissue in a compressed position.

In a further aspect of the present invention, a method is provided for suturing a patient's organ tissue with a laparoscopic surgical clamp for soft tissue repair during a minimally invasive surgical procedure. The method includes providing a laparoscopic surgical clamp. A distal end of the laparoscopic surgical clamp is passed into an opening of a patient's body cavity. The patient's tissue is probed by moving a first set and second set of double jaws of the laparoscopic surgical clamp between an open and closed position. Appropriate tissue is selected for being occluded with the laparoscopic surgical clamp and suturing. The patient's tissue is clamped with the laparoscopic surgical clamp.

At least one elongated suture is passed into the patient's body cavity with a laparoscopic grasper. The elongated suture is threaded through the plurality of fenestration of at least one of an upper portion of the first set and second set of double jaws. The patient's tissue is punctured with the elongated suture. The elongated suture is threaded through the plurality of fenestration of at least one of a lower portion of the first set and second set of double jaws. Each end of the at least one elongated suture is clipped on the upper and lower portions of the first set and second set of double jaws with a clip applicator for securing the patient's tissue in a compressed position.

These and other features and advantages of this invention will become further apparent from the detailed description and accompanying figures that follow. In the figures and description, numerals indicate the various features of the disclosure, like numerals referring to like features throughout both the drawings and the description.

### BRIEF DESCRIPTION OF THE DRAWINGS

FIG. 1 is a perspective view of a laparoscopic surgical clamp used in accordance with the methods of the present invention.

FIG. 2 is a cross-sectional view of the laparoscopic surgical clamp taken along lines 2-2 of FIG. 1.

FIG. 3 is an enlarged perspective view of a first double jaw and a second double jaw of the laparoscopic surgical clamp of FIG. 1.

FIG. 4 is an enlarged perspective view of the second double jaw of the laparoscopic surgical clamp of FIG. 1 having spaces within the jaws for placement of sutures.

FIG. 5 is an enlarged perspective view of a handle assembly of the laparoscopic surgical clamp of FIG. 1.

FIG. 6 is a partial perspective view of a cable system for controlling the jaws of the laparoscopic surgical clamp of FIG. 1.

FIG. 7 is an enlarged partial perspective view of a distal end of exemplary sutures that may be used in combination with the laparoscopic surgical clamp of FIG. 1 according to the methods of the present invention.

FIG. 8A is a perspective side view of suturing method into liver during resection of liver with the laparoscopic surgical clamp in place according to the present invention.

FIG. 8B is a perspective side view of suturing method into liver during post-resection of liver with the suture and clips in place according to the present invention.

FIG. 9 illustrates an elevated perspective view of an open wedge-shaped surgical defect on liver reapposed using the methods of the present invention.

FIG. 9A illustrates an elevated perspective view of the surgical defect on liver partially apposed with the suture and clips in place according to the methods of the present invention.

FIG. 9B illustrates an elevated perspective view of the surgical defect of liver in which the laceration defect is completely apposed according to the methods of the present invention.

FIG. 10 illustrates an elevated perspective view of a post-resectional defect on liver not amenable for closure apposition in which raw edges are left open with hemostasis and partial closure performed by the suturing method of the present invention using a straight suture.

FIG. 10A illustrates an elevated perspective view of an open defect on liver prior to being closed using the suturing method of the present invention.

FIG. 10B illustrates an elevated perspective view of a closed defect on liver according to suturing method of the present invention.

FIG. 11 is an enlarged partial perspective view of a straight suture shown with clip in place at proximal end of suture.

### DETAILED DESCRIPTION OF THE PREFERRED EMBODIMENTS

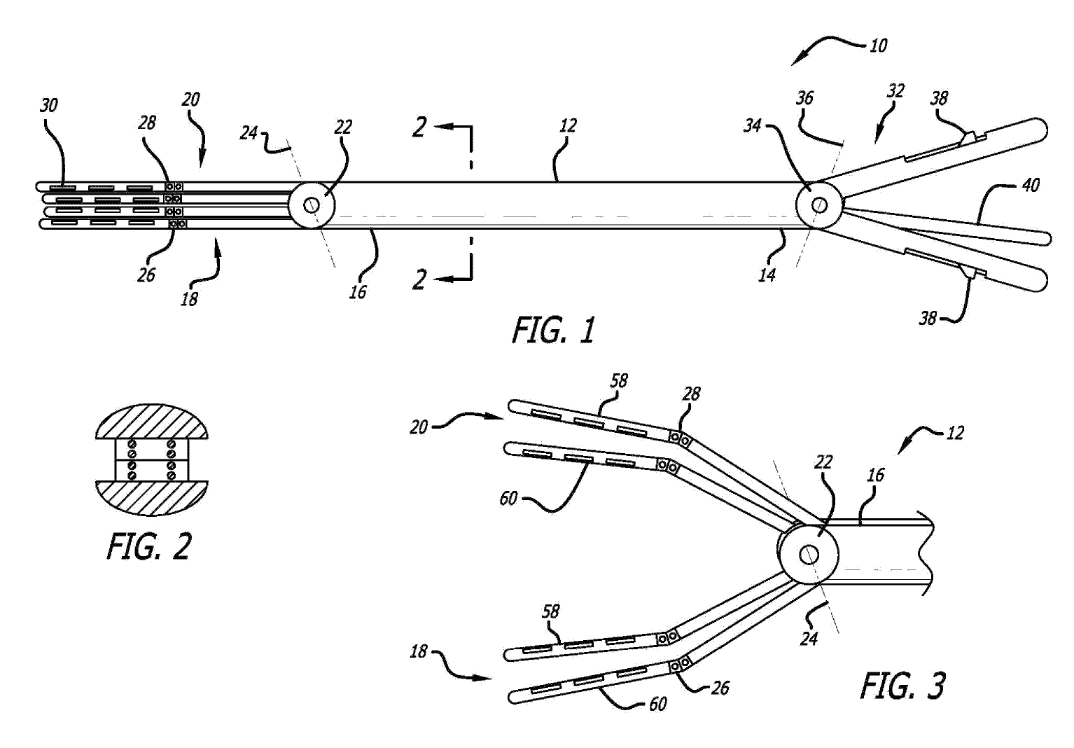

The laparoscopic surgical clamp 10 used in accordance with the methods of the present invention is generally illustrated in FIG. 1. The laparoscopic surgical clamp 10 is particularly configured to be applied to a patient's internal organs, including, but not limited to, the liver, lung, spleen and kidney, during minimally invasive surgical procedures. The laparoscopic surgical clamp 10 includes an elongated shaft 12 having a proximal end 14 and a distal end 16. First set and second set of double jaws 18, 20 are connected to the distal end 16 of the elongated shaft 12 by a pivot 22 and are pivotally moveable relative to one another about a rotational axis 24.

The laparoscopic surgical clamp has the capability of being manipulated into various positions as desired by the surgeon during the minimally invasive surgical procedure. In an opened position, the first set and second set of double jaws 18, 20 are separated and substantially parallel to one another. In a closed position (FIG. 1), the first set and second set of double jaws 18, 20 are adjacent and substantially parallel to one another. In an articulated position (FIG. 3), at least one articulation segment 26, 28 on each jaw of the first set and second set of double jaws 18, 20 is configured such that the jaws may be positioned in a U- or V-shaped configuration. When the first set and second set of double jaws 18, 20 are in the articulated position (FIG. 3), the jaws 18, 20 are not substantially parallel to one another and extend distally relative to the elongated shaft 12.

Referring further to FIG. 1, a plurality of fenestration 30 is formed on at least one jaw of the first set and second set of double jaws 18, 20 of the laparoscopic surgical clamp 10. This allows the surgeon to thread suturing (FIG. 8A) through the first set and second set of double jaws 18, 20 to firmly secure the surgical clamp to a patient's clamped tissue during the minimally invasive surgical procedure.

The laparoscopic surgical clamp further includes a double handle assembly 32 operatively connected to at least one of the first set and second set of double jaws 18, 20 by a fine cable 44 (FIG. 6) such that the jaws 18, 20 may move between the opened and closed positions with ease relative to the motion of the double handle assembly 32. The double handle assembly 32 is connected to the elongated shaft 12 by a pivot 34 and is pivotally movable about a rotational axis 36. The double handle assembly 32 may be connected to the shaft by means other than a pivot that are known in the art.

The double handle assembly is configured to have a switch-type locking mechanism 38, which allows ease of engagement of the first set and second set of double jaws 18, 20 between the opened and closed positions. The double handle assembly 32 also includes an inner handle 40 allowing control of the first set of double jaws 18, 20 into the closed position. The first set and second set of double jaws 18, 20 are capable of being changed to at least one orientation with respect to the shaft 12 operatively connecting the jaws 18, 20 to the double handle assembly 32.

The double handle assembly 32 allows the surgeon to hold the laparoscopic surgical clamp 10 with one hand, open and close the first set and second set of double jaws 18, 20, and apply the locking mechanism 38. The laparoscopic surgical clamp 10 will remain locked as long as the surgeon desires and can be unlocked by the surgeon with ease to readjust position of surgical clamp 10 or remove the surgical clamp 10 from the body cavity. In addition, the pressure applied to the liver parenchyma or other organ parenchyma through the jaws 18, 20 can be adjustable. In the locked position, the double handle assembly 32 will need to be protected and avoid movement to prevent the jaws 18, 20 of the laparoscopic surgical clamp 10 on the solid organ from moving or torquing into undesirable positions. The laparoscopic surgical clamp 10 when properly applied will compress the liver to the point of achieving hemostasis and not cut into liver parenchyma or other organ parenchyma. Tears into the solid organ parenchyma can still occur.

FIG. 2 illustrates a cross-sectional view of the laparoscopic surgical clamp of FIG. 1. The laparoscopic surgical clamp is narrow and elongated when the first set and second set of double jaws are in the closed position (FIG. 1). The first set and second set of double jaws 18, 20 extend laterally from the elongated shaft when in the closed position (FIG. 1).

In FIG. 3, the first set and second set of double jaws 18, 20 of the laparoscopic surgical clamp 10 of FIG. 1 are illustrated in greater detail. The articulation segments 26, 28 of the first set and second set of double jaws 18, 20 are configured such that the jaws may be positioned in a U- or V-shaped configuration.

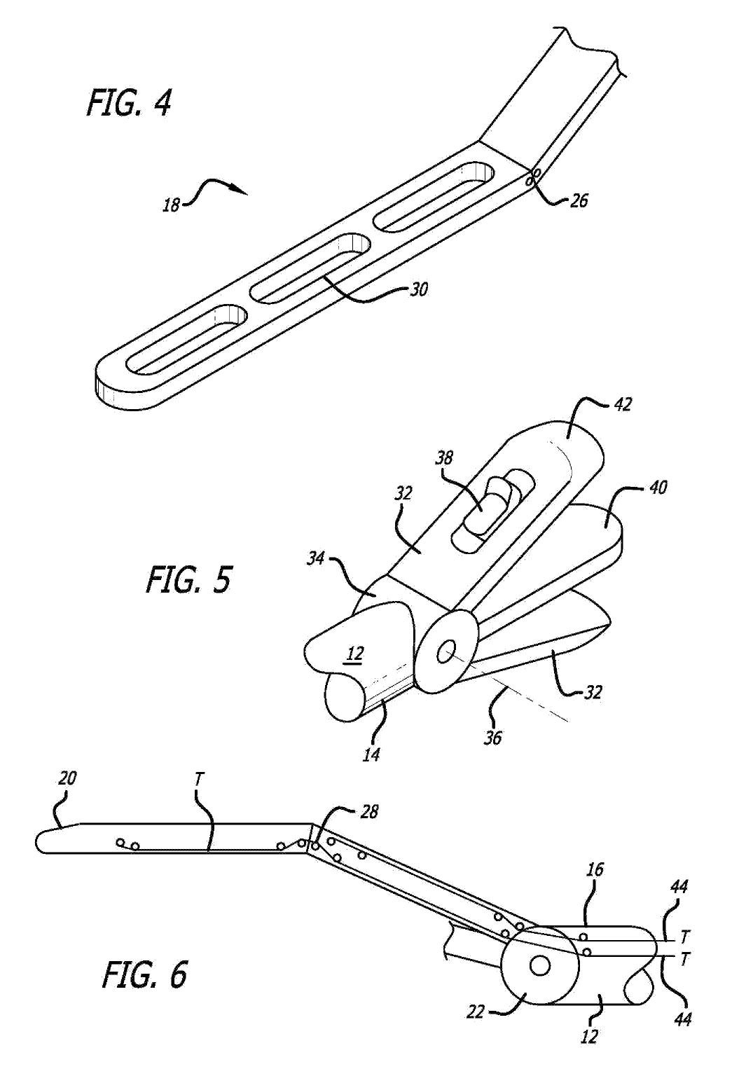

Referring now to FIG. 4, a jaw portion 18 of the first set of double jaws 18 of FIG. 1 is shown in greater detail. The jaw portion includes a plurality of fenestration 30 or open spaces configured into its design such that the surgeon can pass sutures therethrough to secure the surgical clamp to the patient's clamped tissue during the minimally invasive surgical procedure.

FIG. 5 illustrates an enlarged perspective view of the double handle assembly 32 of the laparoscopic surgical clamp 10 of FIG. 1. In one embodiment, the double handle assembly 32 includes a switch-type locking mechanism 38 configured into its design on an outside surface 42 thereof. The locking mechanism 38 is not limited to a switch-type design as other types of locking mechanisms, such as a lever (not shown), may be used in accordance with the methods of the present invention. As previously set forth, the locking mechanism 38 allows ease of engagement of the first set and second set of double jaws 18, 20 between the opened and closed positions. The inner handle 40 allows control of the first set of double jaws 18, 20 into the closed position.

Referring now to FIG. 6, a partial perspective view of a cable system for controlling the jaws 18, 20 of the laparoscopic surgical clamp of FIG. 1 is illustrated in detail. Control of the first set and second set of double jaws 18, 20 is manipulated by a plurality of fine cables 44 having a tension T. Tension of the fine cables 44 is operatively controlled by the double handle assembly 32. Tension applied to the fine cables 44 and the ability to control tension on the cables by the locking mechanism 38 determines the closure pressure on the jaws 18, 20, which in turn determines the tissue pressure resulting in the desired hemostasis. The pressure can be visually gauged by the surgeon as excessive pressure can cause tissue fracture.

In one embodiment, at least one of the plurality of fine cables 44 is connected to the articulated segments 26, 28 of the first set and second set of double jaws 18, 20 to effect both closure and articulation of the jaws 18, 20. Tension is transmitted to the jaws 18, 20 through the plurality of fine cables 44 by applying greater closing or apposing pressure on the double handle assembly 32. The locking mechanism 38 on the double handle assembly 32 is configured to lock in the applied pressure.

The first set and second set of double jaws 18, 20 of the laparoscopic surgical clamp may be aligned, shaped and/or sized according to their intended use depending on such factors as the organ or tissue clamped, surgeon preference, type of surgical procedure involved, and the like. For example, the first set and second set of double jaws 18, 20 may be straight or curved along an axial length thereof to accommodate the curvature of the patient's organ tissue, and long or short. The jaws may be designed such that when directly opposed, they are transversely, horizontally, or vertically aligned with each other. The double row design of the jaws 18, 20 provides the laparoscopic surgical clamp with greater compressibility of the clamped tissue. In further embodiments, depending on the material composition of the first set and second set of double jaws 18, 20, the jaws 18, 20 may be designed to have varying degrees of flexibility and stiffness along their axial lengths thereof such that they are malleable at their distal ends and can be shaped by the surgeon prior to deployment of the laparoscopic surgical clamp.

Once the first set and second set of double jaws 18, 20 are applied and liver resection has begun, the jaws 18, 20 of the laparoscopic surgical clamp 10 can slip off the edge of the liver. Thus, suturing may be necessary to avoid slippage of the first set and second set of double jaws 18, 20. Two or more laparoscopic surgical clamps 10 may be necessary to complete hemostasis on liver to allow resection with minimal blood loss. For example, the application of each surgical clamp 10 isolates the blood supply to the segment occupied by the tumor and the two surgical clamps 10 apposed in a triangular manner isolate a pie shape volume for complete hemostasis. Once the surgical clamp is applied, further compression may be achieved by suturing the jaws 18, 20 closer together to prevent jaw slippage off cut edge of liver or other organs during the minimally invasive surgical procedure.

Two laparoscopic surgical clamps 10 can be used during the minimally invasive surgical procedure in which the surgical clamps 10 are applied from opposing angles for central lesions and large tumors. The laparoscopic surgical clamp 10 of the present invention enables the surgeon to conduct resections of liver lesions of differing sizes and anatomical locations and lesions closer to the major liver vessels. The laparoscopic surgical clamp 10 can also be used on the spleen in a laparoscopic partial splenectomy, or on the kidney, in a laparoscopic partial nephrectomy.

It is contemplated by the present invention that the double jaws 18, 20 can form a U- or V-shaped configuration as earlier disclosed herein. The U-shaped configuration may be further achieved by using two surgical clamps and applying the two jaws 18, 20 at varying angles of deployment. The angling of the jaws 18, 20 requires an additional cable system (not shown) at 90° from the direction of fine cables that will close the jaws 18, 20. The V-shaped configuration may be further achieved by using two surgical clamps 10 coming from two separate trochars (not shown) and converging at a point on the liver parenchyma thereby isolating a V or pie-shape of the liver to be resected.

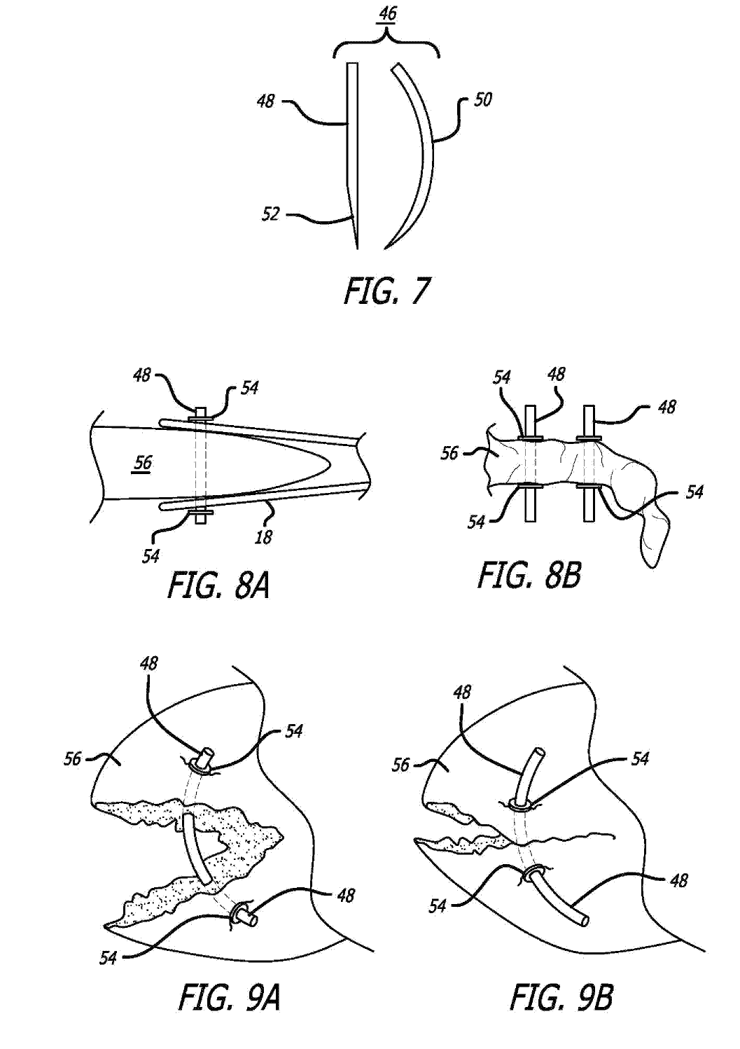

Referring now to FIG. 7 is a distal end of exemplary sutures 46, such as straight 48 or curved 50, which may be used in conjunction with the laparoscopic surgical clamp 10 of FIG. 1 in accordance with the methods of the present invention. The straight suture 48 design can be punctured through the entire thickness of the liver tissue with ease. The curved suture 50 design can be used to apply the suture through the same surface of the liver or other organ and close or compress a liver lesion or other organ tear or appose two liver or other organ surfaces.

In one embodiment, the suture 48 is hollow as a thin tube-like member (FIG. 7) with a pointed distal end 52 to allow puncturing through the liver or other solid organ parenchyma. The suture 48 can be pulled into the body cavity with ease using a laparoscopic grasper. The suture 48 length is variable and can be trimmed to a desired length depending on the particular minimally invasive surgical procedure. The distal suture tip 52 is narrow and solid as in a pointed tip to allow introduction into the liver or other solid organs such as the spleen or kidney as a needle. The suture 48 is capable of puncturing through the entire thickness of the patient's tissue allowing closure and pressure on the same tissue plane. The material of the suture 48 is bio-absorbable, such as a polyglycolic polymer. The measurements of the suture 48 are about 1.5-2 mm in diameter and about 10 cm long with a needle point 52.

The present invention discloses a method of occluding a patient's organ tissue during a minimally invasive surgical procedure. In one embodiment, the method includes providing a laparoscopic surgical clamp 10 as previously disclosed herein and shown in FIGS. 1-6. A distal end 11 of the laparoscopic surgical clamp 10 is passed into an opening of the patient's body cavity. The surgeon probes the patient's tissue by moving the first set and second set of double jaws 18, 20 between the opened and closed positions. The surgeon then selects tissue appropriate for being occluded with the laparoscopic surgical clamp 10 and suturing. The tissue is clamped with the laparoscopic surgical clamp 10. At least one elongated suture 48 is passed into the patient's body cavity with a laparoscopic grasper.

The suture 48 can be passed through the plurality of fenestration 30 of at least one of the first set and second set of double jaws 18, 20 of the laparoscopic surgical clamp 10. In particular, the elongated suture 48 is threaded through the plurality of fenestration 30 of at least one of an upper portion 58 (FIG. 2) of the first set and second set of double jaws 18, 20. The elongated suture 48 punctures through the patient's tissue. The elongated suture 48 is then threaded through the plurality of fenestration 30 of at least one of a lower portion 60 (FIG. 2) of the first set and second set of double jaws 18, 20. The suture 48 may be cut by laparoscopic scissors and be adjusted to a desired length.

Each end of the elongated suture 48 on the upper and lower portions 58, 60 of the first set and second set of double jaws 18, 20 may be clipped 54 by a metal clip applicator, which will compress the round hollow suture flat, and will increase the surface securing the clip 54 at the level desired. The suture 48 end is expandable allowing fixation within the patient's solid organ parenchyma. Further compression may be achieved by reapplying the clip 54 and cutting off the excess length and discarding the previous clip. This maneuver may be applied to both ends of the suture 48 securing the liver or other organ parenchyma into a compressed and fixed position. In a further embodiment, the suture 46 (FIG. 7) may be configured in a curved shape 50 allowing closure and pressure on the same tissue plane.

Once the resection is complete, additional conventional sutures 48 or clips 54 can be applied on the cut end of the vessels and small bile ducts. Once the vascular control of the liver or other organ is satisfactory, the laparoscopic surgical clamp 10 can be disengaged. The suturing remains in place for hemostasis after resection of the patient's tissue and removal of the laparoscopic surgical clamp 10.

FIG. 8A illustrates the laparoscopic surgical clamp 10 in place secured with clips 54 during resection of the liver 56 according to the method of the present invention. FIG. 8B illustrates the suture 48 and clips 54 in place compressing the liver 56 with the surgical clamp 10 removed during post-resection of the liver 56.

Referring now to FIG. 9 is an elevated perspective view of an open wedge-shaped surgical defect on liver 56 reapposed using the methods of the present invention. FIG. 9A illustrates the surgical defect on liver 56 partially apposed with the suture 48 and clips 54 in place. FIG. 9B illustrates the surgical defect of liver 56 in which the laceration defect is completely apposed in accordance with the methods of the present invention.

The present invention further discloses a method of suturing a patient's organ tissue with a laparoscopic surgical clamp for soft tissue repair during a minimally invasive surgical procedure. In one embodiment, the method includes providing a laparoscopic surgical clamp 10 as previously disclosed herein and shown in FIGS. 1-6. A distal end 11 of the laparoscopic surgical clamp 10 is passed into an opening of the patient's body cavity. The surgeon probes the patient's tissue by moving the first set and second set of double jaws 18, 20 between the opened and closed positions. The surgeon then selects tissue appropriate for being repaired with the laparoscopic surgical clamp 10 and suturing. The tissue is clamped with the laparoscopic surgical clamp 10. At least one elongated suture 48 is passed into the patient's body cavity with a laparoscopic grasper.

The elongated suture 48 is threaded through the plurality of fenestration 30 of at least one of an upper portion 58 (FIG. 2) of the first set and second set of double jaws 18, 20. The elongated suture 48 punctures through the patient's tissue. The elongated suture is then threaded through the plurality of fenestration 30 of at least one of a lower portion 60 (FIG. 2) of the first set and second set of double jaws 18, 20. Each end of the elongated suture 48 on the upper and lower portions 58, 60 of the first set and second set of double jaws 18, 20 is clipped with a clip applicator for securing the patient's tissue in a compressed position.

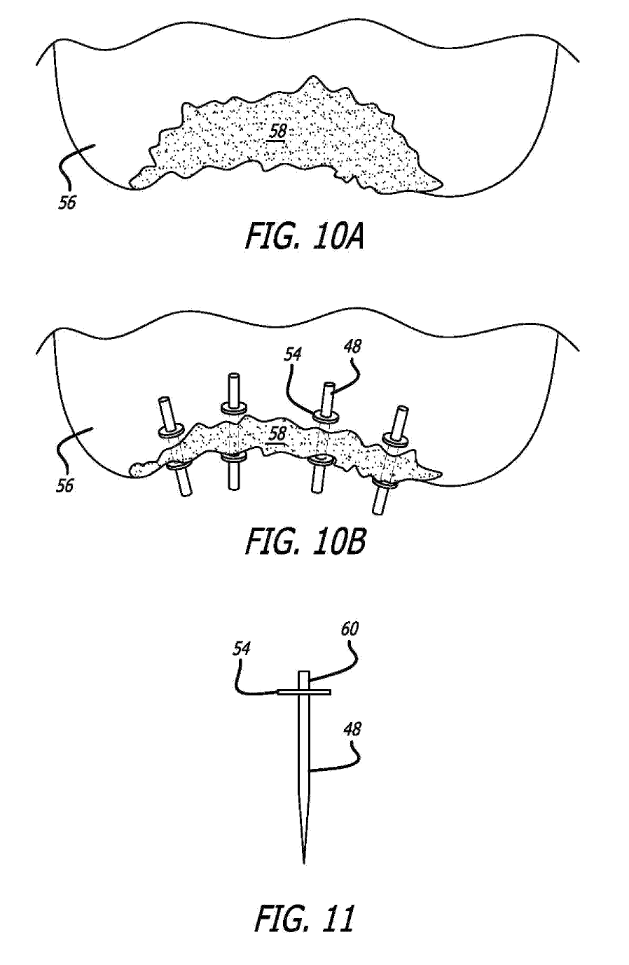

FIG. 10 illustrates an elevated perspective view of a post-resectional defect on liver 56 not amenable for closure apposition in which raw edges 58 are left open with hemostasis and partial closure performed by the suturing method of the present invention using a straight suture 48. FIG. 10A illustrates open defect on liver 56 prior to being closed using the suturing method of the present invention. FIG. 10B illustrates closed defect on liver 56 according to the suturing method of the present invention.

In FIG. 11, straight suture 48 is shown with clip 54 in place at proximal end 60 of suture 48 used in accordance with the suturing method of the present invention.

The laparoscopic surgical clamp used in accordance with the methods of the present invention is uniquely designed to have a low-profile such that it is capable of accessing an abdominal cavity through a 15 mm port during the minimally invasive surgical procedure. In one embodiment, the first set and second set of double jaws 18, 20 may have an operative length of about 12-15 cm. The laparoscopic surgical clamp 10, including the first set and second set of double jaws 18, 20, elongated shaft 12, and the double handle assembly 32, may have an overall operative length of about 37-50 cm.

While the laparoscopic surgical clamp 10 is particularly contemplated for use in laparoscopic or endoscopic minimally invasive surgical procedures, it may also be used in a variety of open surgical procedures, including those requiring occlusion of organs such as the liver, lung, spleen, kidney, and the like.

The laparoscopic surgical clamp 10 may be fabricated from virtually any biocompatible material, including, but not limited to, stainless steel and its alloys, titanium alloys (i.e., nickel-titanium), polymers (i.e., polyethylene and copolymers thereof, and the like), and combinations thereof.

The first set and second set of double jaws 18, 20 may include a removable insert (not shown) on the inside surface of each jaw 18, 20 to cushion the patient's clamped tissue. This prevents the patient's tissue from being damaged or otherwise torn by the first set and second set of double jaws 18, 20 of the laparoscopic surgical clamp during the minimally invasive surgical procedure. The cushion may be formed from various materials known in the art that are compliant and hence will provide a cushioning effect to the clamped tissue, such as those described in U.S. application Ser. No. 10/783,811, filed Feb. 19, 2004, which is incorporated herein by reference in its entirety. The cushion inserts may be attached to the jaws 18, 20 by techniques known in the art, such as those described in the immediately aforementioned U.S. application.

The laparoscopic surgical clamp 10 used in accordance with the methods of the present invention may be either disposable or reusable (i.e., sterilizable). Alternatively, portions of the laparoscopic surgical clamp 10 may be reused and sterilized for subsequent use and other portions may be disposed of for no further use.

Having now described the invention in accordance with the requirements of the patent statutes, those skilled in the art will understand how to make changes and modifications in the present invention to meet their specific requirements or conditions. Such changes and modifications may be made without departing from the scope and spirit of the invention as set forth in the following claims.

What is claimed is:

1. A method of occluding a patient's organ tissue during a minimally invasive surgical procedure, comprising:
providing a laparoscopic surgical clamp, comprising:
an elongated shaft having a proximal end and a distal end;
a first set and a second set of double jaws connected to the distal end of the elongated shaft and moveable relative to one another, the first set and second set of double jaws comprising:
an opened position wherein the first set and second set of double jaws are separated and substantially parallel to one another and a closed position wherein the first set and second set of double jaws are adjacent and substantially parallel to one another;
at least one articulation segment on each jaw of the first set and second set of double jaws such that the first set and second set of double jaws may be positioned in a U- or V-shaped configuration when in an articulated position;
a plurality of fenestration on at least one jaw of the first set and second set of double jaws allowing suturing through the first set and second set of double jaws;
a double handle assembly operatively connected to at least one of the first set and second set of double jaws such that the jaws may move between the opened and closed positions relative to the motion of the double handle assembly;
the double handle assembly having a locking mechanism allowing ease of engagement of the first set and second set of double jaws between the opened and closed positions;
passing a distal end of the laparoscopic surgical clamp into an opening of a patient's body cavity;
probing the patient's tissue by moving the first set and second set of double jaws between the opened and closed positions;
selecting tissue appropriate for being occluded with the laparoscopic surgical clamp and suturing;
clamping the tissue with the laparoscopic surgical clamp;
passing at least one elongated suture into the patient's body cavity with a laparoscopic grasper;
threading the elongated suture through the plurality of fenestration of at least one of an upper portion of the first set and second set of double jaws;
puncturing the patient's tissue with the elongated suture;
threading the elongated suture through the plurality of fenestration of at least one of a lower portion of the first set and second set of double jaws; and
clipping each end of the elongated suture on the upper and lower portions of the first set and second set of double jaws for securing the patient's tissue in a compressed position.

2. The method of claim 1, wherein the suturing remains in place for hemostasis after resection of the patient's tissue and removal of the laparoscopic surgical clamp.

3. The method of claim 1, wherein the suture is configured as a tube-like member having an opening therethrough allowing compression and fixation by a plurality of clips applied across the suture.

4. The method of claim 3, wherein the suture having an opening therethrough may include a curvature to allow closure or compression of a lesion on same surface.

5. The method of claim 1, wherein the suture includes a pointed end for puncturing through the patient's solid organ parenchyma.

6. The method of claim 1, wherein the suture includes a narrow and solid pointed tip such that the suture may be introduced into the patient's tissue as a needle.

7. The method of claim 1, wherein the suture is fabricated from an absorbable material such as a polyglycolic polymer.

8. The method of claim 1, wherein the suture is about 1.5-2 mm in diameter and about 10 cm in length.

9. The method of claim 1, wherein the suture is capable of puncturing through the entire thickness of the patient's tissue allowing closure and pressure on same tissue plane.

10. The method of claim 1, wherein further compression of the suture against the patient's tissue may be achieved by reapplying the clip and cutting off the excess length of the suture and discarding the earlier clip.

11. The method of claim 1, wherein the suture may be cut by laparoscopic scissors and be adjusted to a desired length.

12. The method of claim 1, wherein the suture end is expandable allowing fixation within the parenchyma of the patient's solid organ.

13. The method of claim 1, wherein the suture may be configured in a curved shaped allowing closure and pressure on same tissue plane.

14. The method of claim 1, wherein each end of the elongated suture on the upper and lower portions of the first set and second set of double jaws is clipped with a clip applicator.

15. The method of claim 1, wherein the suturing prevents the surgical clamp from slipping off a cut edge of organ tissue.

16. A method of occluding a patient's organ tissue during a minimally invasive surgical procedure, comprising:
providing a laparoscopic surgical clamp;
passing a distal end of the laparoscopic surgical clamp into an opening of a patient's body cavity;
probing the patient's tissue by moving a first set and second set of double jaws of the laparoscopic surgical clamp between an open and closed position;
selecting tissue appropriate for being occluded with the laparoscopic surgical clamp and suturing;
clamping the tissue with the laparoscopic surgical clamp;
passing at least one elongated suture into the patient's body cavity with a laparoscopic grasper;
threading the elongated suture through a plurality of fenestration of at least one of an upper portion of the first set and second set of double jaws;
puncturing the patient's tissue with the elongated suture;
threading the elongated suture through the plurality of fenestration of at least one of a lower portion of the first set and second set of double jaws; and
clipping each end of the elongated suture on the upper and lower portions of the first set and second set of double jaws with a clip applicator for securing the patient's tissue in a compressed position.

17. A method of suturing a patient's organ tissue with a laparoscopic surgical clamp for soft tissue repair during a minimally invasive surgical procedure, comprising:
providing a laparoscopic surgical clamp, comprising:
an elongated shaft having a proximal end and a distal end;
a first set and a second set of double jaws connected to the distal end of the elongated shaft and moveable relative to one another, the first set and second set of double jaws comprising:
an opened position wherein the first set and second set of double jaws are separated and substantially parallel to one another and a closed position wherein the first set and second set of double jaws are adjacent and substantially parallel to one another;
at least one articulation segment on each jaw of the first set and second set of double jaws such that the first set and second set of double jaws may be positioned in a U- or V-shaped configuration when in an articulated position;
a plurality of fenestration on at least one jaw of the first set and second set of double jaws allowing suturing through the first set and second set of double jaws;
a double handle assembly operatively connected to at least one of the first set and second set of double jaws such that the jaws may move between the opened and closed positions relative to the motion of the double handle assembly;
the double handle assembly having a locking mechanism allowing ease of engagement of the first set and second set of double jaws between the opened and closed positions;
passing a distal end of the laparoscopic surgical clamp into an opening of a patient's body cavity;
probing the patient's tissue by moving the first set and second set of double jaws between the open and closed positions;
selecting tissue appropriate for being occluded with the laparoscopic surgical clamp and suturing;
clamping the tissue with the laparoscopic surgical clamp;
passing at least one elongated suture into the patient's body cavity with a laparoscopic grasper;
threading the elongated suture through the plurality of fenestration of at least one of an upper portion of the first set and second set of double jaws;
puncturing the patient's tissue with the elongated suture;
threading the elongated suture through the plurality of fenestration of at least one of a lower portion of the first set and second set of double jaws; and
clipping each end of the elongated suture on the upper and lower portions of the first set and second set of double jaws with a clip applicator for securing the patient's tissue in a compressed position.
